SEQUENCE LISTING (8 sequences)

SEQ ID NO: 1 - DNA, 20 bp, Artificial Sequence
  Description of Artificial Sequence Synthetic primer
gctggcttccatgagacttc

SEQ ID NO: 2 - DNA, 20 bp, Artificial Sequence
  Description of Artificial Sequence Synthetic primer
agggtgtgatgggatggata

SEQ ID NO: 3 - DNA, 20 bp, Artificial Sequence
  Description of Artificial Sequence Synthetic primer
agtctgttgggggaggaagt

SEQ ID NO: 4 - DNA, 20 bp, Artificial Sequence
  Description of Artificial Sequence Synthetic primer
ccacaaactggtggtctcct

SEQ ID NO: 5 - DNA, 20 bp, Artificial Sequence
  Description of Artificial Sequence Synthetic primer
gaggaagaggaaggcaaacc

SEQ ID NO: 6 - DNA, 20 bp, Artificial Sequence
  Description of Artificial Sequence Synthetic primer
ggcttcttggtgctctcttg

SEQ ID NO: 7 - DNA, 11 bp, Artificial Sequence
  Description of Artificial Sequence Synthetic probe
atggagatgca

SEQ ID NO: 8 - DNA, 20 bp, Artificial Sequence
  Description of Artificial Sequence Synthetic oligonucleotide
gaggaagaggaaggcaaacc

(19) United States
(12) Patent Application Publication
(10) Pub. No.: US 2013/0303394 A1
(43) Pub. Date: Nov. 14, 2013

(54) FLUIDIC CHIPS HAVING SURFACE ENERGY TRAPS

(71) Applicants: Jeff Tza-Huei Wang, Timonium, MD (US); Yi Zhang, Baltimore, MD (US)

(72) Inventors: Jeff Tza-Huei Wang, Timonium, MD (US); Yi Zhang, Baltimore, MD (US)

(73) Assignee: The Johns Hopkins University, Baltimore, MD (US)

(21) Appl. No.: 13/856,320

(22) Filed: Apr. 3, 2013

Related U.S. Application Data
(60) Provisional application No. 61/619,478, filed on Apr. 3, 2012.
(60) Provisional application No. 61/619,481, filed on Apr. 3, 2012.
(60) Provisional application No. 61/676,419, filed on Jul. 27, 2012.

(51) Int. Cl. C12Q 1/68

(52) U.S. Cl. CPC C12Q 1/6876
USPC 506/9; 216/41; 216/45; 435/6.12; 435/287.2

(57) ABSTRACT

A method of using a fluidic device includes providing a fluidic chip having a plurality of surface energy traps; depositing at least one fluid droplet on the fluidic chip, the fluid droplet including magnetic particles suspended therein; and moving at least a portion of the at least one fluid droplet across a surface of the fluidic chip by altering a magnetic interaction applied to the magnetic particles such that the at least one fluid droplet interacts with at least one of the plurality of surface energy traps.

### CROSS-REFERENCE OF RELATED APPLICATION

This application claims priority to U.S. Provisional Application Nos. 61/619,478, filed Apr. 3, 2012; 61/619,481, filed Apr. 3, 2012; and 61/676,419, filed Jul. 27, 2012; the entire contents of all of which are hereby incorporated by reference.

### FEDERAL FUNDING BY THE U.S. GOVERNMENT

This invention was made with Government support of Grant Nos. U54CA151838 and R01CA155305, awarded by the Department of Health and Human Services, The National Institutes of Health (NIH); and Grant Nos. 0546012 and 1033744 awarded by the National Science Foundation (NSF). The U.S. Government has certain rights in this invention.

### BACKGROUND

1. Field of Invention

The field of the currently claimed embodiments of this invention relates to fluidic chips having surface energy traps, methods of producing fluidic chips having surface energy traps, and methods of using fluidic chips having surface energy traps.

2. Discussion of Related Art

Implementing complex bioanalytical assays on fully integrated and scalable micro devices has great potential in key biomedical applications such as point-of-care diagnostics and high-throughput screening (1-5). However, there are significant challenges because the current lab-on-a-chip (LOC) devices require intricate microfluidic networks to transfer and process the biological samples and reagents. Most such microfluidic chips are implemented in a continuous flow format, requiring multifaceted fluidic architectures, components such as pumps and valves, and an external fluid interface to carry out complex bioassays. To address the challenges of these channel-based, continuous flow systems, there is increasing interest in developing droplet-based microfluidic systems (6-9). Droplet microfluidic platforms enable simple, stand-alone, and reconfigurable fluidic architectures, in which discrete droplets function as vessels for material storage and transfer in the bioanalytical assays.

Diverse mechanisms have been used for droplet actuation, including electrowetting (10-13), magnetic force (14-17), surface acoustic wave (18), and dielectrophoresis (19). Of these, electrowetting is most widely used because it facilitates the core fluid operations of dispensing, splitting, and transport. Nonetheless, such a wide range of droplet operations requires a closed or two-plate configuration, in which droplets are tightly sandwiched between two substrates patterned with electrodes, resulting in a restricted operating liquid volume (100 s nl-1 μl) (6, 8). This small assay volume may be impractical for assays that require high sensitivity, such as PCR-based detection of infectious agents due to statistical sampling considerations (8). Furthermore, electrowetting alone is limited to liquid handling, and cannot be used to manipulate the solid materials used in heterogeneous assays. Usually a secondary mechanism, such as magnetic forces or dielectrophoresis, is needed for particle handling (20-23).

In contrast, magnetic actuation uses an external magnetic field to manipulate droplets, containing magnetizable particles (MPs), through solid-liquid interfacial interactions. Since MPs also serve as a solid phase for molecule absorption and separation, magnetic actuation provides a promising approach to implementing bioanalytical assays in digital microfluidic systems. However, magnetic actuation alone performs only a limited set of simple liquid operations, thus significantly hindering its applicability in complex assays. For example, liquid dispensing, which is a universal process required for sample aliquoting, serial dilution, and droplet splitting for parallel and multiplexed reactions, has not been achieved on any magnetic droplet platforms. There thus remains a need for improved microfluidic chips and systems.

### SUMMARY

A method of producing a fluidic chip according to some embodiments of the current invention includes providing a substrate having a first surface with a first free energy; coating the first surface of the substrate with a layer of a second material having a surface with a second free energy; arranging a shadow mask over the layer of the second material, the shadow mask defining a pattern of apertures; and etching portions of the layer of the second material through the pattern of apertures to expose underlying portions of the first surface of the substrate such that the fluidic chip has a pattern of localized first free energy regions surrounded by the second material to provide surface energy traps for droplets of fluid. The first free energy differs in value from the second free energy.

A method of using a fluidic device according to some embodiments of the current invention includes providing a fluidic chip having a plurality of surface energy traps; depositing at least one fluid droplet on the fluidic chip, the fluid droplet including magnetic particles suspended therein; and moving at least a portion of the at least one fluid droplet across a surface of the fluidic chip by altering a magnetic interaction applied to the magnetic particles such that the at least one fluid droplet interacts with at least one of the plurality of surface energy traps.

A fluidic chip according to some embodiments of the current invention includes a glass substrate, and a layer of polytetrafluoroethylene on a surface of the glass substrate. The layer of polytetrafluoroethylene defines a pattern of apertures therethrough to expose surface portions of the glass substrate so as to provide a pattern of surface energy traps.

### BRIEF DESCRIPTION OF THE DRAWINGS

Further objectives and advantages will become apparent from a consideration of the description, drawings, and examples.

FIG. 1 is a schematic illustration to help explain methods of producing fluidic chips according to some embodiments of the current invention.

FIG. 2 is a schematic illustration of a fluidic chip according to an embodiment of the current invention.

FIGS. 3A-3D help explain methods of producing shadow masks and fluidic chips according to some embodiments of the current invention.

FIG. 4 is a schematic illustration that helps explain methods of producing fluidic chips using modular shadow mask sections according to some embodiments of the current invention.

FIG. 5 is a schematic illustration of a fluidic chip that can be used according to some embodiments of the current invention.

FIGS. 6A-6C demonstration SET assisted magnetic droplet manipulation according to some embodiments of the current invention. a) SET assisted droplet immobilization and MPs splitting. Droplets can move freely in regions with low surface energy. Left-hand droplet moves to merge with right-hand droplet and the merged droplet is immobilized by the SET, which facilitates the splitting of the MPs plug from the droplet. b) and c) Droplet metering and aliquoting b) in air and c) in oil environment. Small SETs are not able to immobilize the entire droplet. Instead, an aliquot is metered and held back by the SET. The volume of the aliquotted daughter droplet is determined by the size of the SET.

FIGS. 7A-7C provide a conceptual illustration of SETs and magnetic droplet manipulation according to an embodiment of the current invention. (A) The shadow mask made of SU8 photoresist, lithographically fabricated by a lift-off process, is used to pattern SETs on the TEFLON-coated glass substrate. (B) SETs are patterned by selectively etching the TEFLON AF nanofilm with O₂ plasma passing through perforations in the SU8 shadow mask. (C) The TEFLON AF nanofilm provides a low energy surface for (I) droplet transport and (II) droplet fusion. SETs are used to (III) pin down the contact line for extraction of MPs from the droplet and (IV) withhold portions of the liquid to generate new daughter droplets.

FIGS. 8A-8E illustrate full-range droplet manipulation on a glass substrate with SETs according to an embodiment of the current invention. MPs are shown in black. (A) Droplet transport. The left-hand droplet is transported towards the trapped right-hand droplet that serves as a spatial reference. (B) Droplets fusion and mixing. Once the two droplets are brought into close proximity, they spontaneously fuse, creating into a bigger droplet. The MPs oscillate in the fused droplet, thus speeding up mixing. (C) MPs extraction. The SET holds the droplet in position, but allows the MPs to pass through, thereby facilitating their extraction. (D) Droplets dispensing. Compared to droplet and MPs size, SETs are not large enough to trap the entire droplets, and thus only a small portion of the liquid is held back by each SET. (E) Cross-chip droplet transfer. SETs provide surface tension high enough to overcome gravity, and can selectively pick up target droplets and transfer them from one platform to another.

FIGS. 9A and 9B illustrate liquid dispensing using SETs. (A) The volume dispensed by SETs is determined by size of SETs. For droplets generated on the same chip with SETs of the same size, the coefficient of variation (CV) in volume was approximately 3%. For droplets dispensed on different chips, the CV was larger, approximately 10%, due to chip-to-chip variation. (B) Droplet microarray generated using 3 different sizes of SETs.

FIGS. 10A-10H show an example of an integrated platform for multiplexed genetic detection on a SETs-enabled magnetic droplet platform according to an embodiment of the current invention. (A-F) Demonstration of the integrated process with food color to aid visualization. (A) The layout of chip with buffers pre-deposited on the designated locations. (B) The crude sample is first mixed with the lysis/binding buffer droplet and MPs (Not shown here). Cells in the sample are lysed and DNA molecules bind to the MPs surface. The SETs assist the MPs extraction from the lysis/binding buffer droplet. MPs move through washing buffer droplets to rinse off the contaminants. (C) The eluent containing DNA molecules are made into 3 aliquots by SETs. (D) The PCR reagent droplet is merged with the eluent aliquot. (E) The SETs assist the extraction of the MPs from the reaction mixture. (F) Samples are ready for PCR. (G) Real time amplification curve of PCR on the SETs-enabled droplet platform monitored with customized miniaturized optical detection system (FIG. S3). (H) Multiplexed detection of TP53, HER2 and RSF1 genetic biomarkers on the SETs-enabled droplet platform. DNA isolated from the sample is dispensed into 3 aliquots by SETs. Dark spots indicate positive amplification.

FIGS. 11A-11F illustrate FlipDrop array according to another embodiment of the current invention. (A) N targets are made into an N by N array by moving N target droplets over an array of SETs. Each target droplet generates N aliquots. (B) N probes are made into the array of the same size of the top array, but in the perpendicular direction. (C) The probe array is flipped down onto the target array, thus allowing droplet merging. (D) N samples and N probes lead to the formation of a total of N² unique combinations with a single flip. (E) Fluorescence scans of the FlipDrop array for DNA sensing. (F) The FRET factors are calculated based on the mean fluorescent intensities, and are interpolated and presented in a heat map.

FIGS. 12A-12F provide a conceptual illustration of making serial dilution with SETs according to another embodiment of the current invention. (A) Step 1: The parent droplet containing fluorescein stock solution is dragged over a serial of SETs of varying sizes to dispense daughter droplets. (B) Step 2: The equal volume dilution buffer droplets are moved to merge with the daughter droplets containing fluorescein. (C) Step 3: MPs are removed from the dilution series, leaving droplets containing fluorescein in the dilution series. (D) The expected concentration of the fluorescein is plotted as a function of the measured concentration in log-log scale. The slope of the linear regression is close to 1, suggesting the actual dilution factor is as expected. (E-F) Serial dilutions of ampicillin are generated to measure the antibiotics susceptibility of E. coli. (E) The growth of the resistant strain is not affected by ampicillin. (F) The growth of the susceptible strain is inhibited by ampicillin with a minimal inhibitory concentration of 2 μg/mL.

FIGS. 13A-13D show (A) SETs operation phase diagram with constant droplet volume (10 μL), and variations in magnetic particle input and SET diameter. (B) SETs operation phase diagram with constant SET size (2 mm diameter), and variation in magnetic particle input and droplet volume. (C) 3D phase diagram of SETs enabled magnetic droplet operation. (D) Force diagram of the droplet being stretched by the MPs moving over the SETs.

FIGS. 14A-14I show droplet operations on SETs enabled magnetic digital platform. Each panel represents an experiment with a fixed droplet volume. (A) 5μL, (B) 10 μL, (C) 20 μL, (D) 30 μL, (E) 40 μL, (F) 50 μL, (G) 60 μL, (H) 80 μL, and (I) 100 μL. The results are summarized and qualitatively represented in a 3D phase diagram shown in FIG. 8.

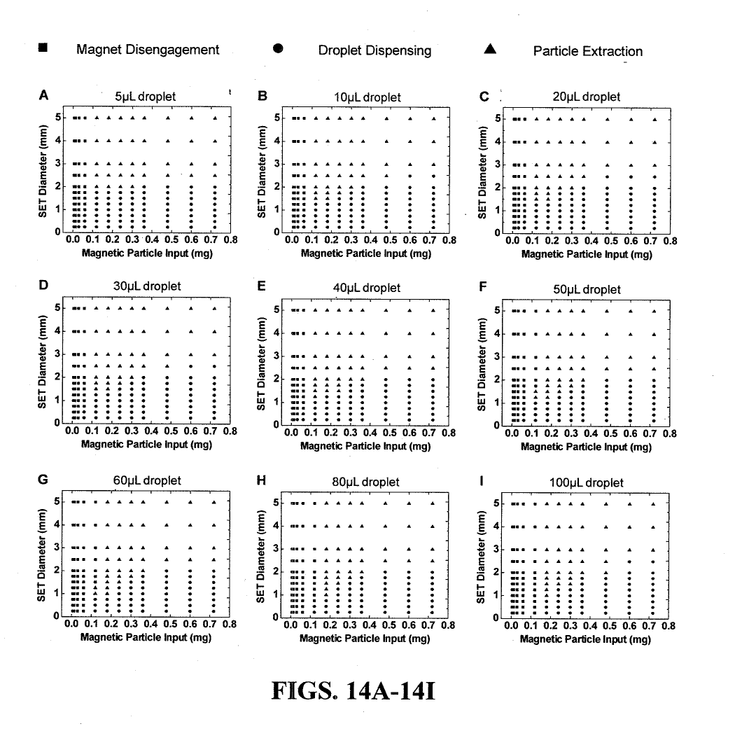

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

TEFLON AF is a type of fluorinated polymer material commonly used to create hydrophobic/oleophobic surfaces for various types of applications. Many applications require the TEFLON AF coating in designated areas instead of creating a homogeneous coating covering the entire substrate. By doing so, one can modify the surface with desired combinations of hydrophobic and hydrophilic regions. However, patterning the TEFLON AF using a lithographic approach is not straightforward because the TEFLON coated substrate is not wettable by photoresist due to the low surface energy. To address the problem according to some embodiments of the current invention, we provide a novel fabrication process to precisely define the pattern of TEFLON AF using reactive ion etching through an SU8 shadow mask. The SU8 shadow mask is fabricated using a novel liftoff process using the TEFLON AF as a sacrificial layer. Using the SU8 shadow mask, the entire TEFLON AF patterning process can be completed in less than 2 minutes. Compared to traditional lithography, our process significantly reduces the fabrication time, thereby improving the throughput and yield. Some embodiments of the current invention can also provide a rapid prototyping method for patterning different designs on TEFLON AF coated substrates using a movable SU8 shadow mask. The movable SU8 shadow mask resembles the movable type used in printing. Desired patterns can be made into small SU8 masks that function as tablets used in the printing. By arranging the pre-fabricated shadow mask tablets in specific orders and combinations, we do not need to re-create a new SU8 shadow mask for every new design. This novel fabrication technique is the first and so far the best approach the precisely and reliably pattern the TEFLON AF film coated on a solid substrate.

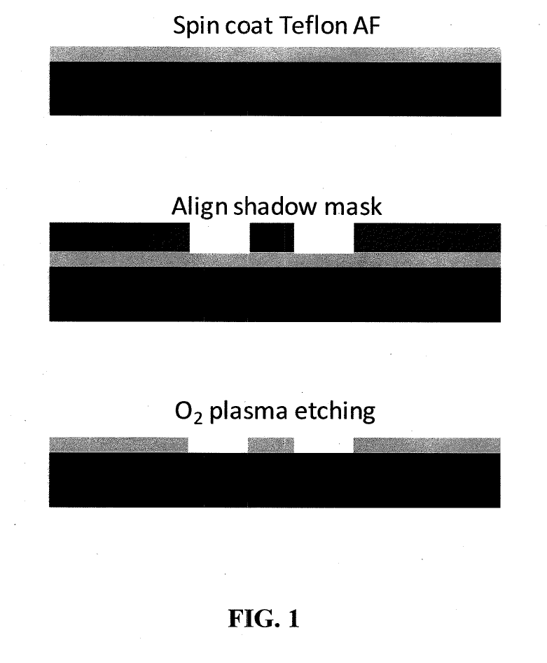

A method of producing a fluidic chip according to an embodiment of the current invention is illustrated schematically in FIG. 1. The method includes providing a substrate having a first surface with a first free energy, coating the first surface of the substrate with a layer of a second material having a surface with a second free energy, arranging a shadow mask over the layer of the second material, the shadow mask defining a pattern of apertures, and etching portions of the layer of the second material through the pattern of apertures to expose underlying portions of the first surface of the substrate such that the fluidic chip has a pattern of localized first free energy regions surrounded by the second material to provide surface energy traps for droplets of fluid. The first free energy differs in value from the second free energy. In FIG. 1, the substrate is the lower structure. In this example, the layer of the second material is TEFLON AF; however, the general concepts of the current invention are not limited to this example. In addition, the etching is done by reactive ion etching, more specifically, O₂ plasma etching, in the example of FIG. 1, which is not required in all embodiments of the current invention.

In some embodiments, the above-noted steps can be repeated a plurality of times, sequentially, in that order, to produce a corresponding plurality of fluidic chips. In some embodiments, the shadow mask can be reused for producing the plurality of fluidic chips. This can reduce the time, cost and complexity of producing fluidic chips.

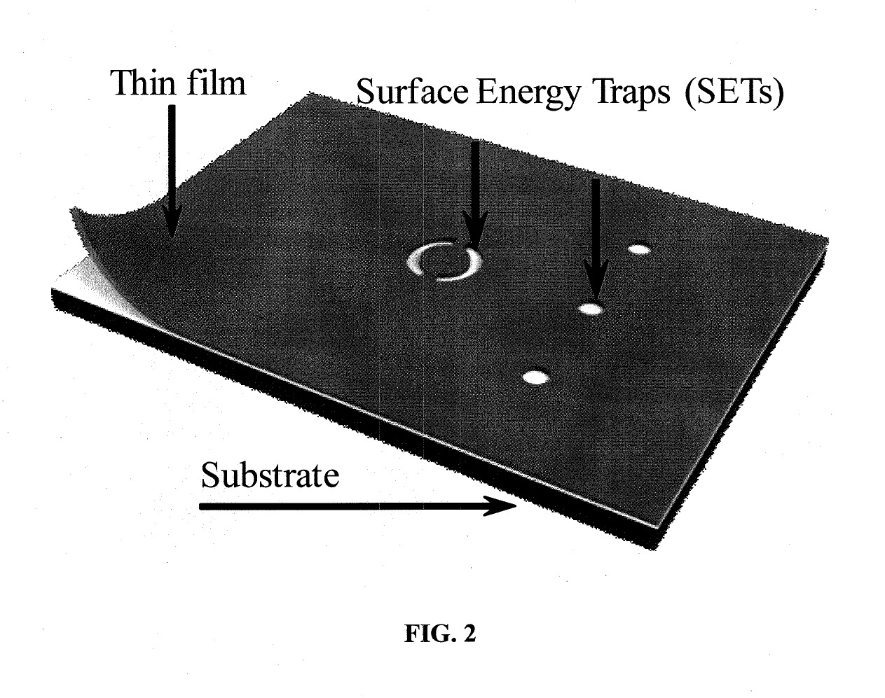

FIG. 2 is a schematic illustration of a fluidic chip according to an embodiment of the current invention. IT shows an example of three circular SETs and two semi-annular apertures that form a different type of SET.

In some embodiments, the second free energy is greater than the first free energy. In some embodiments, the first surface of the substrate can be a hydrophilic surface and the layer of second material can be a hydrophobic surface, for example. In some embodiments, the substrate can be a glass substrate and the layer of second material can be a layer of polytetrafluoroethylene (e.g., TEFLON AF). However, the general concepts of the current invention are not limited to these examples.

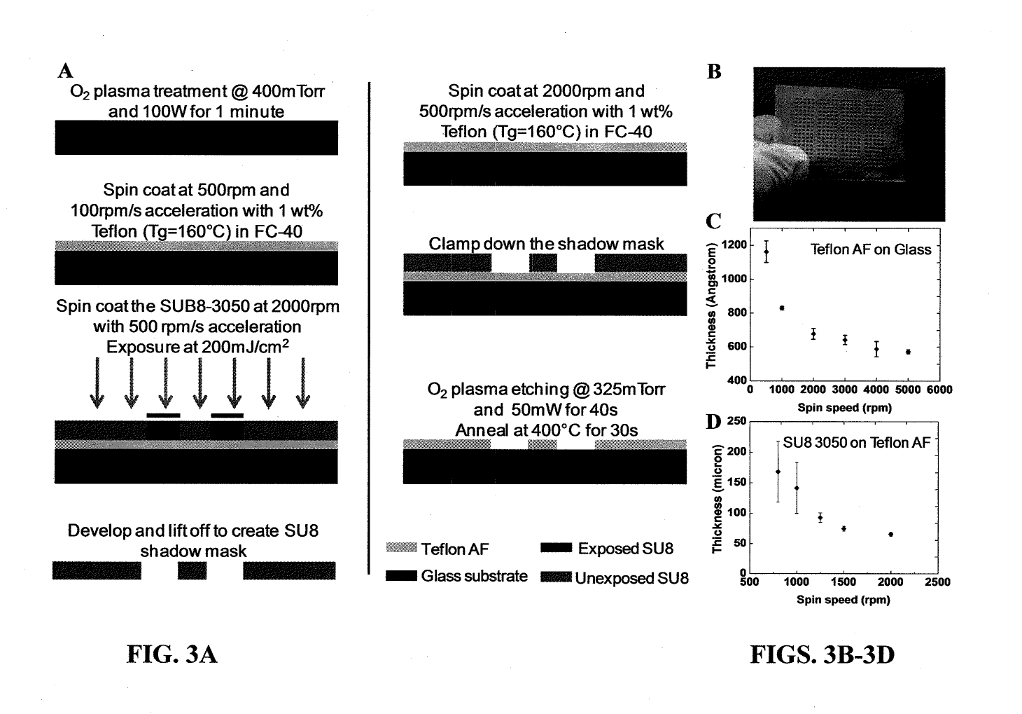

In some embodiments, the shadow mask can be made from a photoresist material, such as, but not limited to, SU8. FIG. 3A provides a schematic illustration that also includes a method of producing the shadow mask. It should be kept in mind that once the shadow mask is produced, it can be re-used many times. In the example of FIG. 3A, the shadow mask is produced by a photolithographic process.

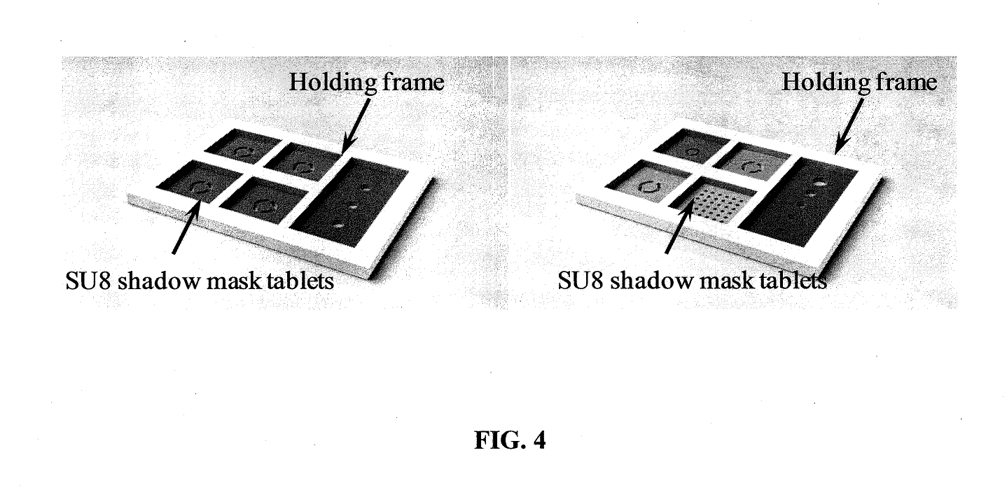

In some embodiments, the shadow mask can include a plurality of apertures patterns such that different aperture patterns can be selected to provide selectable patterns of surface energy traps. FIG. 4 shows an example in which preformed sections can be combined in different patterns. For example, the left hand side shows one combination of four preformed square section and a rectangular section combined for one possible chip design. The right hand side provides a different combination of four square and one rectangular sections combined for another design. One should understand, in analogy to type setting, there is a very large number of combinations that could be made with a limited number of pre-formed sections.

SU8 Shadow Masks Fabrication and SETs Patterning.

The following describes an example according to an embodiment of the current invention in detail. The general concepts of the current invention are not limited to this example. The SU8 shadow mask was lithographically defined with a layer of TEFLON AF as the sacrificial layer in the lift off process (FIG. 3A). The glass substrate was first cleaned with O₂ plasma. 1 wt % TEFLON AF (T_{g}=160° C.) dissolved fluorinated oil (Fluorinert FC-40) was then spun on the substrate at 500 rpm, resulting in a layer of ˜120 nm in thickness. The thickness of TEFLON AF layer was estimated by measuring the spectral reflectance using Filmetrics F20. The relationship between TEFLON AF film thickness and spin speed was plotted in FIG. 3C. The TEFLON AF coated glass substrate was then treated with O₂ plasma for 5 s to improve the wettability for better adhesion. SU8 3050 was spin coated at 2000 rpm. The thickness of SU8 3050 on O₂ plasma treated TEFLON AF versus spin speed was shown in FIG. 3D. After exposure with a dosage of 200 mJ/cm², the SU8 membrane was lifted off in SU8 developer, cleaned with isopropanol alcohol (IPA), and dried with compressed air (FIG. 3B). To pattern SET, 1 wt % TEFLON AF was coated on a piece of grade 1 glass coverslip at 2000 rpm. The SU8 shadow mask was then laminated on the coverslip and fixed with clamps. By subjecting the substrate to O₂ plasma at 325 mTorr and 50 mW for 40 s, the exposed TEFLON AF was removed while the area protected by the SU8 membrane remained intact. We noticed that TEFLON AF film disintegrated when it came in contact with water after the O₂ plasma etching process. Therefore, the last step was to bake the coverslip at 400° C. for 30 s in order to anneal the TEFLON AF and promote its adhesion to the glass. The entire SETs patterning process was completed in 2 min.

The SETs fabrication can allow us to create patterns from TEFLON AF within a matter of minutes. As a result of the use of SU8 photoresist as an etching shadow mask, the pattern of the shadow mask can be easily and precisely defined using traditional lithography. To lift off the SU8 membrane from the substrate, a sacrificial layer is placed in between them; We discovered that TEFLON AF works much better as a sacrificial layer than positive photoresists. With TEFLON AF, the SU8 membrane is easily detached from the substrate in the developer. In contrast, with positive photoresists as the sacrificial layer, it requires vigorous sonication in the acetone bath to lift off SU8, which causes the SU8 membrane to swell and deform. The shadow mask patterning offers a rapid and reliable way of making SETs. Once the shadow mask is ready, it takes less than 2 minutes to complete the entire SETs patterning process. The SU8 shadow mask is reusable and durable. This approach can significantly decrease the turnaround the time and cost of producing the fluidic chips. This can be important for a disposable platform for point-of-care bioassays, for example.

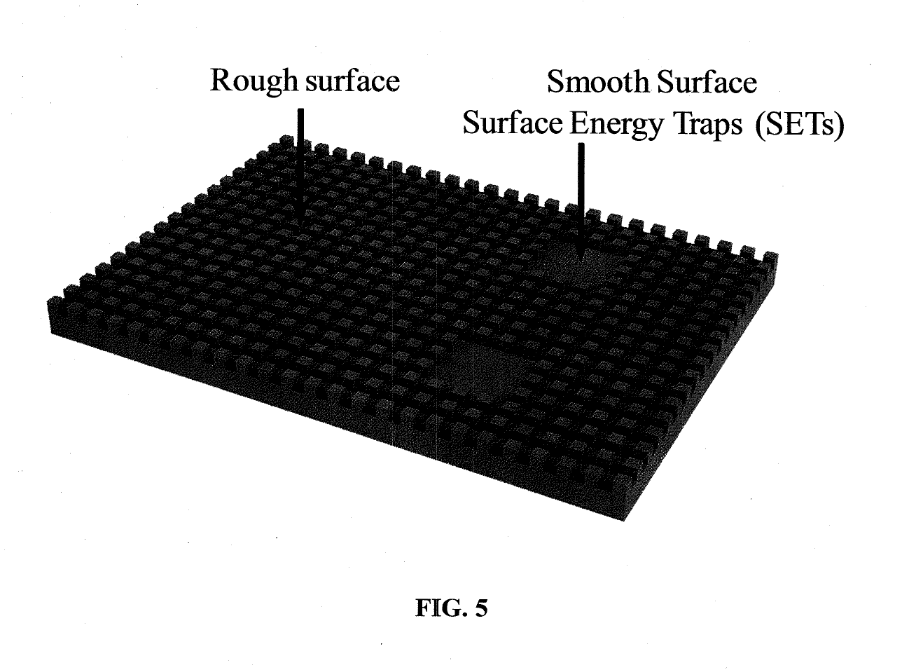

FIG. 5 shows an example of an alternative fluidic chip that can be used according to embodiments of the current invention.

In operation, the surface energy traps (SETs) assisted droplet platform can provide highly complex droplet manipulation, which can greatly improve the functionality of fluidic chips for point-of-care applications. On digital microfluidic platforms, material transfers and reactions can be realized through moving, merging and splitting the droplets with various actuation methods (Y. Zhang, et al., 2011, Lab Chip, 11, 398-406; J. Pipper, et al., 2007, Nat. Med., 13, 1259-1263; J. Pipper, et al., 2008, Angew. Chem., 47, 3900-3904; C. K. Cho, et al., 2003, J. Microelectromechanical Systems, 12, 70-80; Z. Guttenberg, et al., 2005, Lab Chip, 5, 308-317). Magnetic actuation can be particularly useful for point-of-care applications because the magnetic particles used not only function as droplet actuator, but also serve as a substrate to facilitate solid phase extraction (SPE). We have earlier demonstrated sample-to-answer molecular diagnostics on a magnetic droplet platform patterned with surface topographic features to facilitate droplet manipulation (Y. Zhang, et al., 2011, Lab Chip, 11, 398-406; U.S. application Ser. No. 13/745,511; and PCT/US2011/045363, the entire contents of which are incorporated herein by reference). Nonetheless, it still lacks of functional components for complex droplet operation, such as fluid dispensing and aliquoting, which so far is only achievable through electrowetting actuation (C. K. Cho, et al., 2003, J. Microelectromechanical Systems, 12, 70-80). SETs according to some embodiments of the current invention can facilitate complex magnetic droplet handling.

The SETs are high-energy islands surrounded by regions of surface with low free energy. They function by altering the surface wetting property of the substrate. SETs interact with droplets by pinning down the 3-phase contact line thereby trapping the liquid within their boundaries. There are two major approaches to construct surface energy traps. The first method is to create a heterogeneous surface by selectively patterning a thin film with low surface free energy on a substrate with high surface free energy, or vice versa (for example, as in FIG. 2). The second method is to create energy difference on a homogeneous surface by inducing surface roughness using micro- and nano-structures on the substrate (for example, as in FIG. 5). Although consisting of the same material, regions on the substrate with different roughness present different apparent surface free energies, which can be utilized to construct SETs.

An embodiment of the current invention provides a specific example of creating SETs through patterning a TEFLON AF film on a glass substrate and the applications of SETs in various molecular assays. In the example, SETs are patterned by O₂ plasma etching through a SU8 shadow mask, which is lithography defined and lifted-off from a sacrificial layer (FIG. 3A). The shadow mask protects the TEFLON AF nanofilm and meanwhile exposes the SETs regions. The unexposed regions maintain their low-energy coating while the exposed regions are etched and rendered hydrophilic. As a result, SETs possess high surface energy and trap liquid within their boundaries.

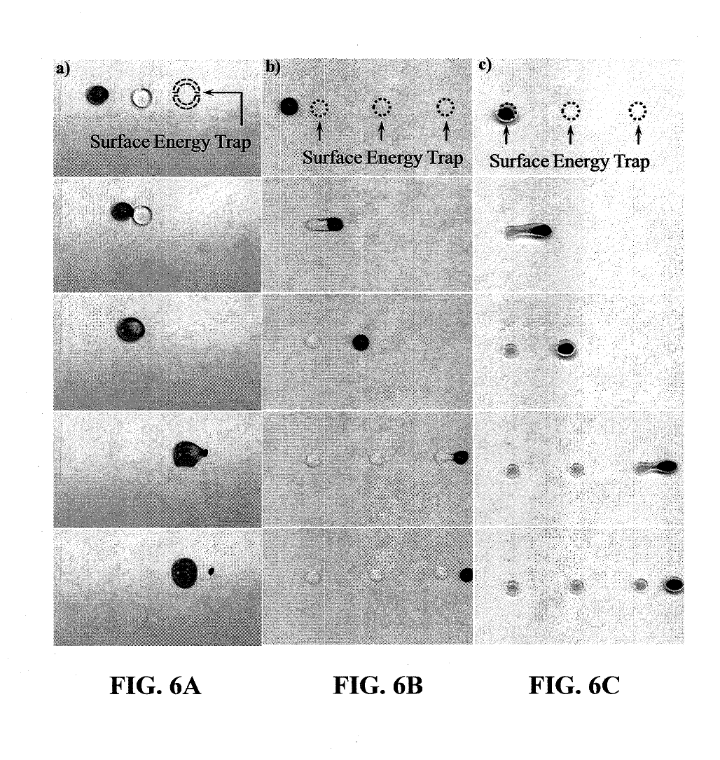

The platform can allow common droplet operation including droplet moving and merging. In FIG. 6A, the left-hand droplet is moved to merge with the right-hand droplet using magnetic actuation with magnetic particles (MPs). SETs are first demonstrated for easy MPs splitting. The SET holds the merged droplet in position whereas the MPs plug overcomes the surface tension and splits from the droplet (FIG. 6A). In addition, droplets of pre-determined volumes can be metered and aliquoted from the parent droplet using SETs (FIGS. 6B-6C). By adjusting the size of the SET, the surface tension along the SET contact line becomes weaker than the capillary force around the MPs plug. As a result, MPs plug does not split from the droplet. Instead, a daughter droplet is metered and aliquoted from the parent droplet. The SET does not only operate in air (FIG. 6B) but also in oil (FIG. 6C), for broader possible applications. The actuation can be done with permanent and/or electromagnets, such as, but not limited to the systems and methods of U.S. application Ser. No. 13/745,511; and PCT/US2011/045363, the entire contents of which are incorporated herein by reference.

Accordingly, a method of using a fluidic device according to some embodiments of the current invention includes providing a fluidic chip having a plurality of surface energy traps; depositing at least one fluid droplet on the fluidic chip, the fluid droplet comprising magnetic particles suspended therein; and moving at least a portion of the at least one fluid droplet across a surface of said fluidic chip by altering a magnetic interaction applied to the magnetic particles such that the at least one fluid droplet interacts with at least one of the plurality of surface energy traps. The moving the at least a portion of the at least one fluid droplet across a surface of the fluidic chip can include moving it to a selected surface energy trap. The surface energy trap can be empty or can have a second fluid droplet such that the moving causes the at least a portion of the at least one fluid droplet to merge with the second droplet.

The moving at least a portion of the at least one fluid droplet across a surface of the fluidic chip can include moving a portion of the at least one fluid droplet out of a surface energy trap while leaving a portion of the at least one fluid droplet in the surface energy trap. The moving at least a portion of the at least one fluid droplet across a surface of the fluidic chip can include moving a portion of the at least one fluid droplet out of a surface energy trap while leaving a portion of the at least one fluid droplet in the surface energy trap to provide splitting of the at least one fluid droplet. The moving at least a portion of the at least one fluid droplet across a surface of the fluidic chip can include moving the at least one fluid droplet across a plurality of surface energy traps to split the at least one fluid droplet a plurality of times leaving sub-droplets at each of the plurality of surface energy traps.

The following examples describe some embodiments and some applications in more detail. However, the broad concepts of the current invention are not limited to the particular examples.

### EXAMPLES

As noted above, magnetic actuation uses an external magnetic field to manipulate droplets, containing magnetizable particles (MPs), through solid-liquid interfacial interactions. Since MPs also serve as a solid phase for molecule absorption and separation, magnetic actuation can provide a useful approach to implementing bioanalytical assays in digital microfluidic systems. However, magnetic actuation alone performs only a limited set of simple liquid operations, thus significantly hindering its applicability in complex assays. For example, liquid dispensing, which is a universal process required for sample aliquoting, serial dilution, and droplet splitting for parallel and multiplexed reactions, has not been previously achieved on any conventional magnetic droplet platforms.

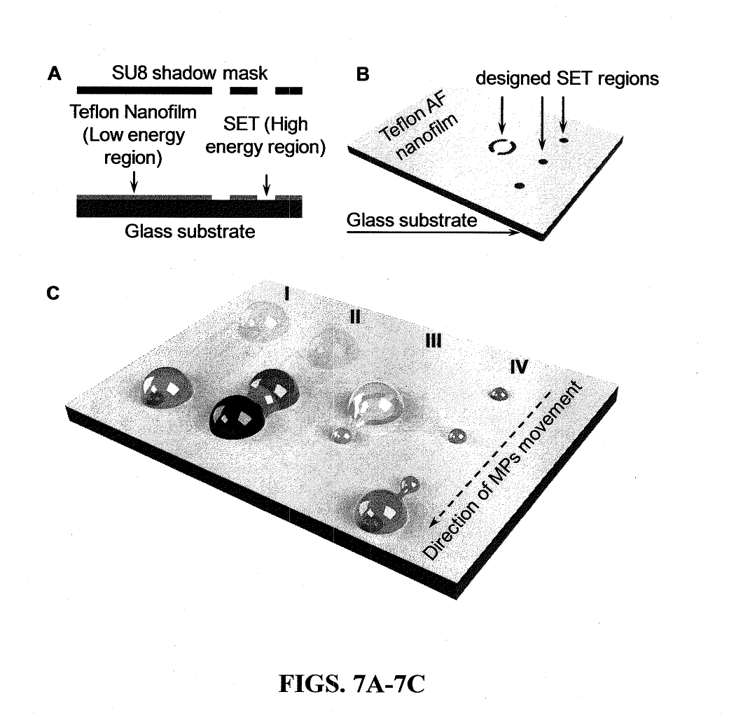

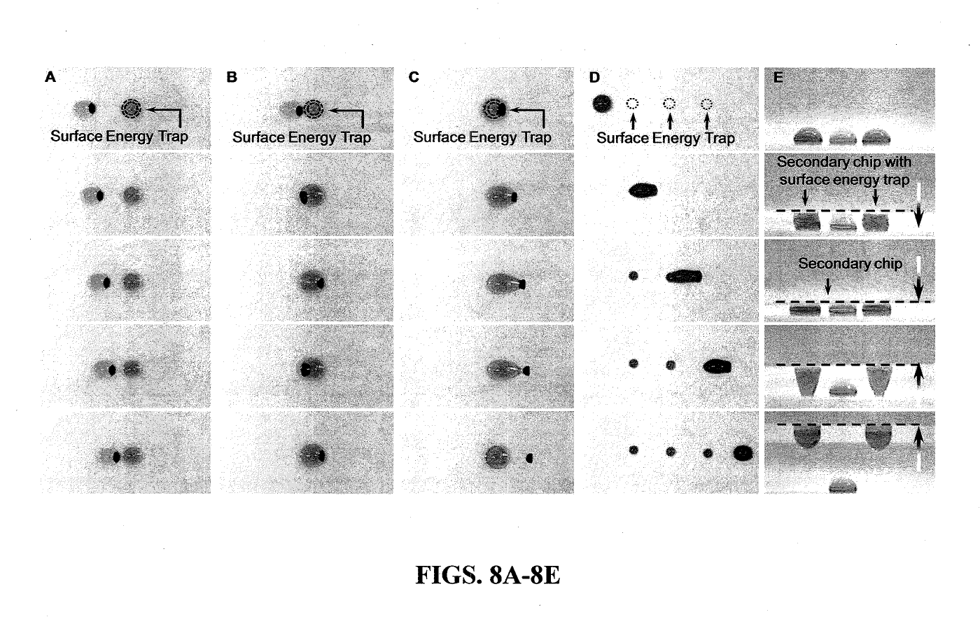

Some embodiments of the current invention can provide a surface energy traps (SETs)-based magnetic droplet manipulation platform that enables a full range of fluid operations. A SET is an etched area of high surface energy on a substrate that is coated with a low surface energy film (FIG. 7A, 7B). By selectively patterning SETs of varying geometries on the substrate, its wettability can be controlled, thereby facilitating comprehensive magnetic droplet manipulations, including droplet dispensing, transport, fusion, particle extraction, and cross-chip droplet transfer (FIG. 7C and FIGS. 8A-8E). The design of the SETs can yield a magnetic digital microfluidic platform capable of complex assays, which could not be achieved by traditional droplet systems. We have demonstrated the versatility of our new platform in three applications: (i) a multiplexed genetic biomarker detection chip fully integrated with sample preparation and PCR, (ii) a combination droplet array named “FlipDrop” for DNA sensing, and (iii) a droplet based serial dilution chip for a bacteria antibiotic susceptibility test. These are some particular examples and are not intended to limit the broad concepts of the current invention.

SETs are fabricated by etching, through a SU8 shadow mask, onto a pre-deposited TEFLON AF film (FIG. 7A, 7B). The fabrication workflow (detailed in “Materials and Methods” below) is now summarized chronologically. The lithographically fabricated shadow mask, used to define the SETs patterns, was made of SU8 photoresist. The TEFLON AF film was spin-coated onto the glass coverslip, which served as rigid substrate, to produce a low-energy surface that allowed droplet movement. Then the SU8 shadow mask, including perforations for the designated SETs, was laminated on top of the TEFLON AF film. Only the areas of the film not protected by the shadow mask were etched when subsequently exposed to O₂ plasma. The SETs were formed by stripping off the exposed TEFLON AF film to unveil the high surface energy glass substrate underneath. The one-step SETs patterning was completed within 3 minutes, and the SU8 shadow mask was reusable.

### Results

### Full-Range Droplet Operation Using SETs.

Droplet manipulation was demonstrated on the SETs-enabled magnetic digital microfluidic platform. The MPs were added to droplets, which had been colored with food dye for ease of visualization on the TEFLON surface (FIG. 8A-8E). By moving a magnet beneath the substrate, the MPs formed a plug and dragged the left droplet (8A), travelling freely on the low-energy regions of the TEFLON film, and transferring the material in the left droplet to pre-designated locations where the right droplet sat (FIG. 8A). When two droplets were brought into close proximity of each other, they fused together, thus mixing their materials and allowing reactions to occur (FIG. 8B).

The extraction of MPs was facilitated by large SETs which immobilized the entire droplet by pinning down the contact line. MPs continued travelling until they broke the surface tension, and separated themselves from the droplet (FIG. 8C). MP extraction is an intricate process, which depends on the interplay between magnetic force, capillary force, and frictional force (24). The operating conditions, such as droplet volume, MPs amount, and magnet moving speed, require fine-tuning within a narrow range to extract MPs without droplet splitting or magnet disengagement. Although early work used physical energy barriers to restrict droplet movement and facilitate MPs extraction (16, 25), in our new platform, the SETs restrict droplet movement by pinning down its contact line, which is far more efficient. Meanwhile, the magnetic force is strong enough to pull the MPs away from the SETs, leaving behind only the droplet.

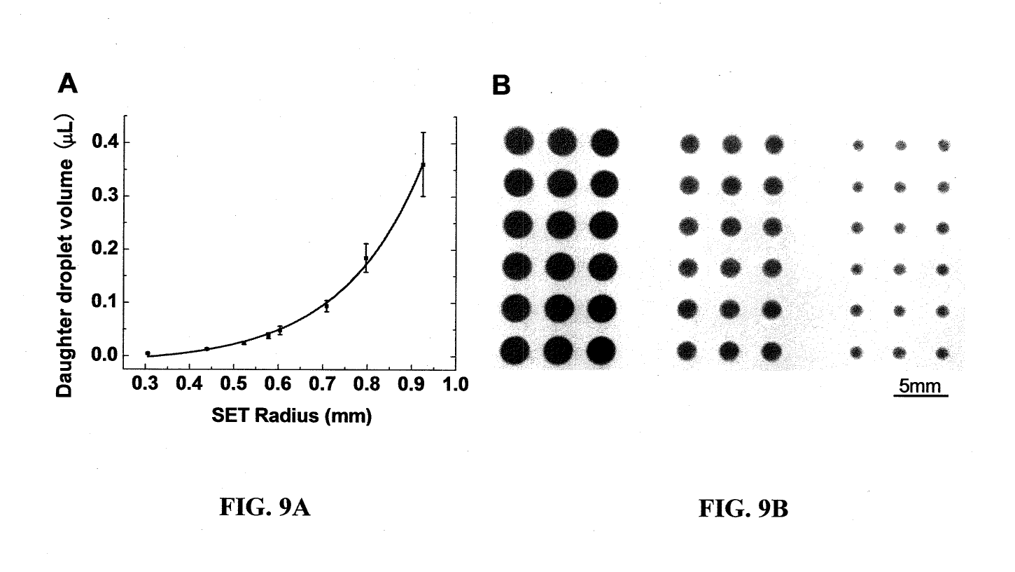

Our method for droplet splitting, allowing liquid to be dispensed and new droplets to be generated, takes advantage of the fact that small SETs withhold only the minority of the liquid, while majority of the droplet escapes from SETs together with MPs (FIG. 8D). Droplets containing fluorescein were dispensed using SETs of various sizes, and their volumes were estimated based on the fluorescent intensities (Equation 1). We show that the volume of the daughter droplet was determined by the size of the SET (FIG. 9A).

Droplets dispensed by SETs exhibited high uniformity. For droplets generated on the same chip with SETs of the same size, the coefficient of variation (CV) in volume was approximately 3%. For droplets dispensed on different chips, the CV was larger, approximately 10%, due to chip-to-chip variation (FIG. 9A). Nonetheless, this method enables the dispensing of sub-microliter volumes on a magnetic digital microfluidic platform with reasonable accuracy. Its simplicity and accuracy provides a rapid and reliable method of generating droplet microarrays on an open droplet platform (FIG. 9B). To further test the applicability of dispensing daughter droplets, we successfully dispensed MPs-containing droplets when they were submerged in mineral oil. This can be a significant advantage because mineral oil is often included in droplet based assays to prevent droplet evaporation during transportation and reactions.

Existing digital microfluidic systems perform all droplet operations on a single chip, and thus the number of droplets is limited by the size of the chip. This restriction is problematic because on-chip applications increasingly demand a high degree of parallelization, and it is impractical to perform all the procedures on a size-limited, single chip. A solution to this problem can be offered by SETs, which allow droplets to be transferred between different chips. A secondary chip was patterned with SETs in specific locations corresponding to those of the droplets on the primary chip which were to be transferred. When the secondary SETs were brought into contact with the sessile droplets on the primary chip, the secondary SETs overcame gravity and picked up the droplets from the primary chip. The large surface tension provided by SETs enabled the complete transfer from chip to chip. (FIG. 8E).

### SETs Applications

Fully-Integrated Multiplex Genetic Detection.

SETs of diverse sizes and functions were arranged on a fully integrated device capable of sample-to-answer, multiplexed genetic detection, from crude samples. Each SET on the device held a specific buffer for the assay, and MPs were used to transfer and combine materials and reagents.

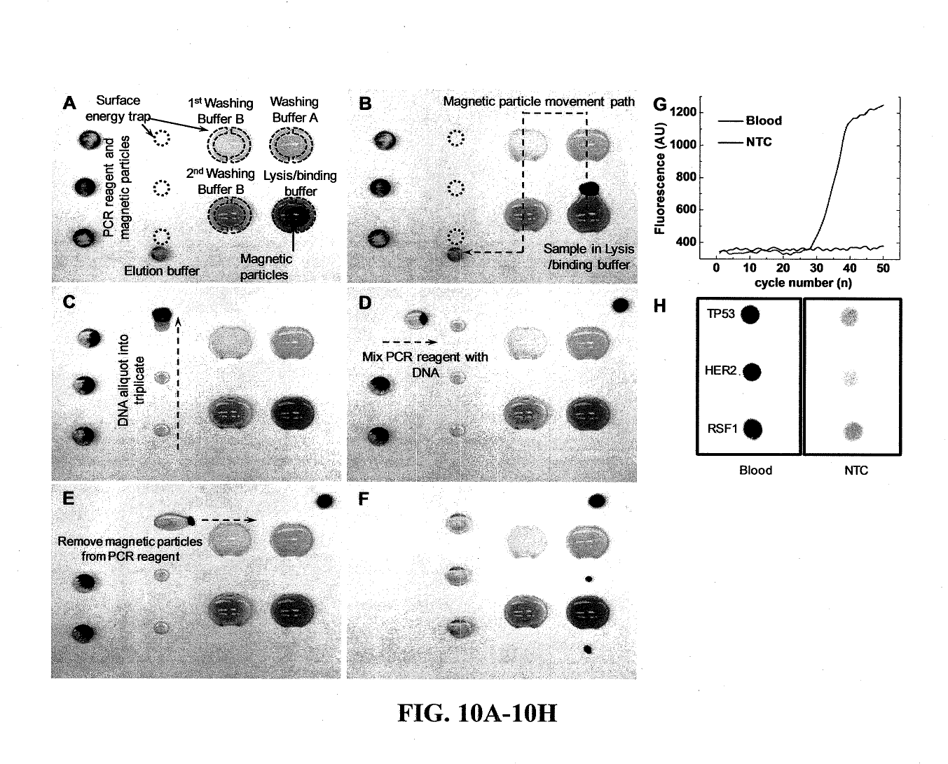

Here we demonstrated the entire process using food color to aid visualization (FIGS. 10A-10H). There were a total of 3 stages involved. First, the platform isolated DNA from whole blood using MPs-based solid phase extraction (FIGS. 10A and 10B). Second, the isolated DNA was dispensed into multiple aliquots using SETs (FIG. 10C). Last, DNA aliquots were mixed with droplets containing gene-specific PCR reagents, and the reaction droplets were subjected to thermal cycling (FIG. 10D-10F).

In the first stage, buffers required for DNA isolation, including the lysis/binding buffer and washing buffers, were held in position by 4 large SETs on the right side of the chip (FIG. 10A). Whole human blood was incubated with the silica coated. MPs and lysis/binding buffer, in which cells were lysed, and DNA adsorbed onto the silica surface. After incubation, MPs were extracted from the lysis/binding buffer droplet with the assistance of SETs (FIG. 10B). MPs were then moved through washing buffer A droplet to remove any carryover contaminants. After that, MPs moved through two other washing buffer droplets in a similar fashion, for further rinsing (FIG. 10B). The 4 SETs-immobilized buffer droplets facilitated MPs extraction, thus greatly improving the performance of DNA extraction on the magnetic droplet platform.

In the second stage, the MPs with surface-bound DNA were incubated with the elution buffer, and the DNA molecules detached from MPs. To detect multiple biomarkers, the eluent containing isolated DNA was split into three aliquots by moving the droplet with MPs over three small SETs (FIG. 10C).

Last, three smaller PCR buffer droplets, each containing a pair of unique primers specific to a different biomarker, were driven by MPs to merge with the three aliquots (FIG. 10D). Subsequently, MPs were removed from the mixture droplets which were held in place by the large SETs (FIG. 10E). The three reaction mixture droplets on the left side of the chip were then placed on a commercial flatbed thermal cycler and subjected to thermal cycling for PCR (FIG. 10F).

The SETs-enabled droplet PCR was monitored in real time using a customized, miniaturized, fluorescence detection system. The detector of the optical system uses a lock-in configuration that allows fluorescence to be measured in ambient light. We used the portable optical system for real time PCR detection of the genetic biomarker RSF1, after performing DNA extraction from whole blood. We successfully identified RSF1 in the blood sample, but not in the no-template control (NTC) (FIG. 10G). Multiplexed biomarker detection was achieved by fluorescence scan, which showed stronger signals in human whole blood than in NTC, indicating successfully identification of all three genes (TP53, HER2 and RSF1) in blood samples (FIG. 10H). All primer and probe sequences are provided in Table 1.

TABLE 1
Primer and probe sequences.

| Primer | Sequence |
|---|---|
| TP53 forward | 5′-GCTGGCTTCCATGAGACTTC-3′ |
| TP53 reverse | 5′-AGGGTGTGATGGGATGGATA-3′ |
| HER2 forward | 5′-AGTCTGTTGGGGGAGGAAGT-3′ |
| HER2 reverse | 5′-CCACAAACTGGTGGTCTCCT-3′ |
| RSF1 forward | 5′-GAGGAAGAGGAAGGCAAACC-3′ |
| RSF1 reverse | 5′-GGCTTCTTGGTGCTCTCTTG-3′ |
| RSF1 probe | 5′-/FAM/ACAATGCTC/ZEN/ATGGAGATGCA/ |
| | Iowa Black/-3′ |
| QD-FRET | 5′-/biotin/GAGGAAGAGGAAGGCAAACC/ |
| | Cy5/-3′ |

FlipDrop: Combination Droplet Array for DNA Sensing.

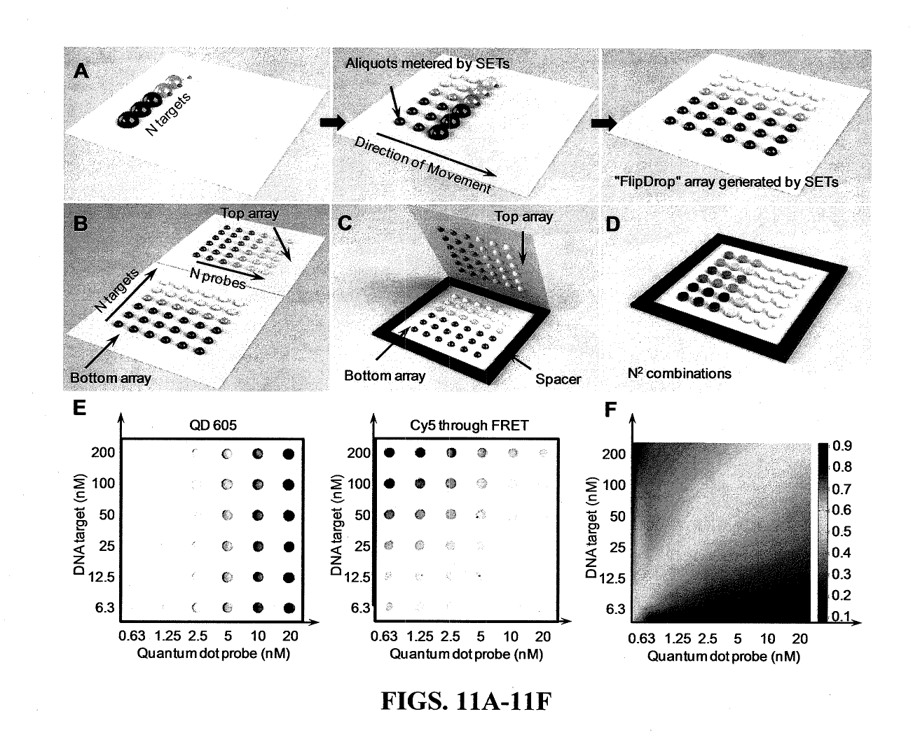

We have developed a combination droplet platform by using droplet arrays that are quickly and reliably generated by SETs with a secondary SET device to transfer and mix droplets between chips. Named “FlipDrop”, this 2-array combination platform can be useful for high throughput screening because of its ease of development and use. With N different targets and N different probes, FlipDrop is able to generate N² combination mixtures of targets and probes with a simple flip (FIG. 11A-11D).

On the bottom array, N droplets, each containing a different target, travel in parallel over a series of N SETs. In so doing, each of the target droplets dispenses an aliquot on each of the N SETs in a single row (FIG. 11A). The same process is repeated on the top array, but with the droplets moving in a perpendicular direction, to dispense a single target on each of the N sets in a single column (FIG. 11B). The top array is then flipped down to combine with the bottom array so that every droplet on the top array is merged with its corresponding droplet on the bottom array (FIG. 11C). As a result, each merged droplet represents a unique target-probe combination, and a total of N² combinations are achieved through a single flip (FIG. 11D). The “FlipDrop” platform provides the “world-to-chip”interface (26, 27) that prepares many combination samples from few inputs, which can be useful for high throughput screening.

To demonstrate the functionality of FlipDrop, we used as a model system, a DNA sensing assay based on quantum dot fluorescence resonance energy transfer (QD-FRET) (28-30). The bottom array carried labeled DNA fragments diluted into 6 different concentrations (The DNA oligonucleotide was labeled with biotin at the 5′ terminal and with Cy5 at the 3′ terminal). The top array was created with streptavidin-coated quantum dots (QDs) with maximal emission at 605 nm (QD 605), also in 6 different concentrations. Once flipped, the droplets on both arrays merged into 36 unique combinations. The labeled DNA fragments self-assembled onto the QDs surface via biotin-streptavidin interaction, which brought the Cy5, the FRET acceptor, into the vicinity of the QDs which served as the FRET donor. The presence of the DNA fragments was detected through the increased emission of Cy5, accompanied by the decreased intensity of QDs as a result of the energy transfer. The corresponding FRET factors were calculated (Equation 2) based on average QDs and Cy5 intensities through FRET, both of which were acquired using a fluorescence scan (FIG. 11E). The results were interpolated and presented in a heat map (FIG. 11F), where the relative quantity of the labeled DNA to QDs determined the FRET factor. A higher DNA to QDs (FRET acceptor to donor) ratio led to larger the FRET factor, and vice versa. Samples along the diagonal exhibited equal FRET factors despite markedly different concentrations because the DNA to QDs ratio remained the same.

Making Serial Dilution for Bacterial Antibiotic Susceptibility Measurement.

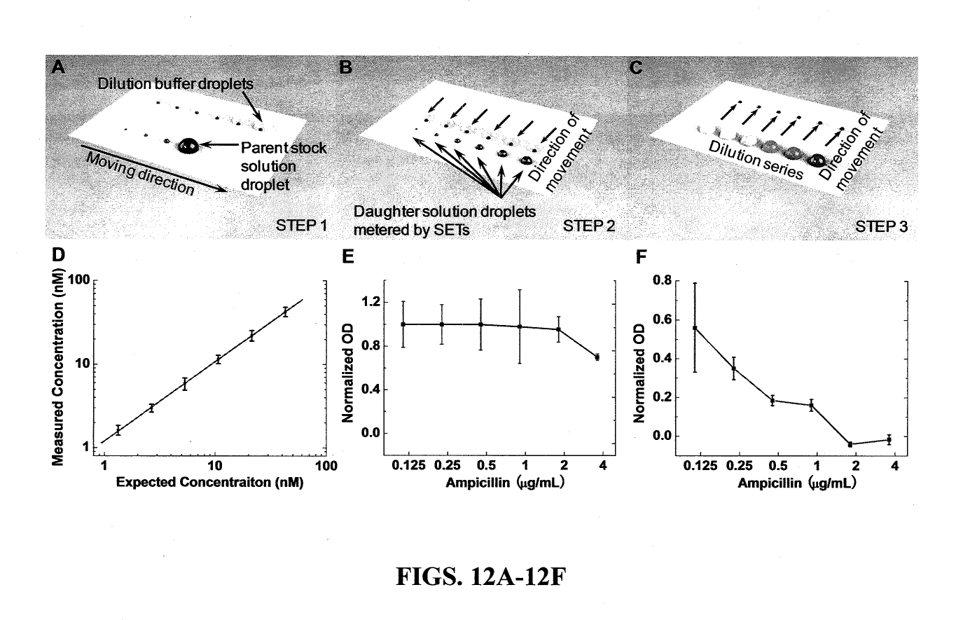

SETs can provide a new strategy for creating a microliter dilution series because they dispense droplets of specified volumes (FIG. 12A-12C). A series of SETs of increasing sizes were fabricated, where the diameter of each SET was calculated based on the dilution factor and the volume of the dilution droplets according to Equation 3 and FIG. 9A. A single stock solution droplet was dragged over the SETs, which dispensed specific volume daughter droplets as dictated by the size of each SET (FIG. 12A). The equal volume dilution buffer droplets were then moved from the TEFLON surface to merge with the metered stock solution (FIG. 12B). Finally, the MPs were removed, leaving only a dilution series in microliter volumes (FIG. 12C). We demonstrate this by using a SET device to create a dilution series of fluorescein with a dilution factor of 2. The measured fluorescein concentrations were plotted against the expected concentrations, and resulted in a slope of 0.95 (FIG. 12D). This indicates a very close match between the measured and expected concentrations, since a slope of 1.0 would indicate an exact match.

To measure the susceptibility of bacteria to antibiotics, we created a two-fold dilution series of ampicillin on a SETs platform. Two strains of Escherichia coli, one resistant to ampicillin and one susceptible to ampicillin, were separately cultured in ampicillin-containing droplets. The final bacteria densities were then measured and plotted against ampicillin concentration. The resistant strain was not affected by the antibiotics, and showed high growth rate regardless the ampicillin concentration (FIG. 12E). In contrast, the susceptible strain had slowed bacteria growth, with a minimum inhibitory concentration of approximately 2 μg/mL (FIG. 12F). The results from the SET's platform agreed well with bench-top experiments in which the dilution series and bacterial culture were performed in a tube.

### DISCUSSION

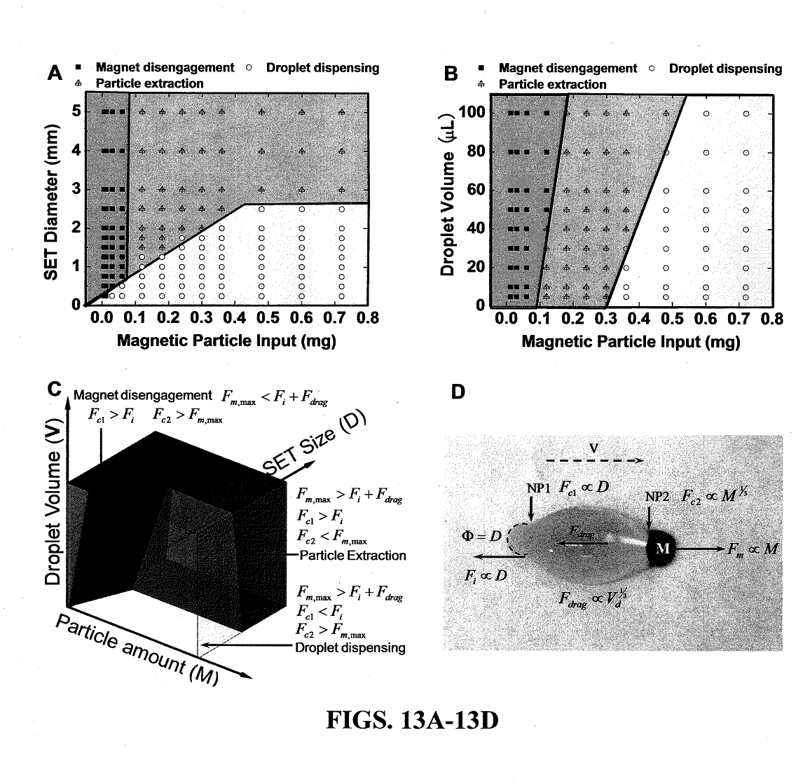

As the MPs pull a droplet over a SET, one of three phenomena are observed, (i) particle extraction, (ii) droplet dispensing, and (iii) magnet disengagement (FIG. 13A-13C). In particle extraction, the entire droplet is immobilized by the SET, while the MPs travel with the magnet and are extracted from the droplet. In droplet dispensing, the SET only holds a small portion of liquid within its boundary, and majority portion of the droplet travels with the MPs, leaving behind a daughter droplet. In magnet disengagement, the SET contains the entire droplet as well as MPs as they disengage from the moving magnet.

To be able to predict which of these three phenomena will occur, we need to understand the governing dynamics. SETs function by immobilizing the liquid through contact line pinning. When MPs are pulled from the SET, they stretch and deform the droplet, leading to the formation of one necking point around the SET (NP1) and one around the MP plug (NP2) (FIG. 13D). As the MPs continue traveling away from the SET, the radius of curvature at the two necking points gradually decreases to a negative value, causing breakage at these points. Breakage at NP2 results in particle extraction, breakage at NP1 results in droplet dispensing, and either necking point breaks in the case of magnet disengagement. Whether the necking points break and which necking point breaks first, depends on the MPs amount, SET size, and droplet volume. Simply speaking, the SET and the MPs engage in a tug-of-war with the droplet until one of the necking points yields.

The phenomenon can be explained by the interaction between four elements: the magnetic force, the surface tension around SET, the capillary forces around the MPs plug and SETs, and the drag force imposed on the droplet (FIG. 13D). The magnetic force Fₘ along the travel direction is proportional to the amount of MPs M (24). At a constant speed, the drag force F_{drag} is approximately proportional to the base radius of the droplet, or V_{d}^{1/3} where V_{d} is the volume of the droplet, assuming the droplet is hemispherical (24). The surface tension Fᵢ around the SET is proportional to its diameter D.

The unique interactions of the four elements that produce particle extraction, droplet dispensing, and magnet disengagement are now described. In particle extraction, NP2 breaks first when Fₘ is greater than the combined force of F_{drag} and Fᵢ, and greater than the capillary force F_{c2} at NP2. Under these conditions, the MPs can overcome the surface tension and split from the droplet (FIG. 13D). In droplet dispensing, NP1 breaks first when Fₘ exceeds the combined force of F_{drag} and Fᵢ, and the capillary force around the SET F_{c1} is smaller than Fᵢ. Meanwhile, Fₘ is smaller than the capillary force F_{c2} at NP2 in order for MPs to remain in the droplet. In magnet disengagement, Fₘ is smaller than F_{c2}, and smaller than the combined force of F_{drag} and Fᵢ. Similarly, the capillary force Fₐ at NP1 is greater than Fᵢ. As a result, the droplet is immobilized by the SET, and MPs are contained within the droplet, in which case neither the droplet nor the MPs can move with the magnet.

With a fixed droplet volume (FIG. 13A), large SETs stabilize NP1, and hold the droplet in position until NP2 breaks, leading to particle extraction. In comparison, large amount of MPs stabilize NP2, and drag the entire droplet until NP1 breaks, leaving behind a small amount of liquid on the SET. Magnet disengagement is observed with small amounts of MPs, in which case Fₘ is too weak for the MPs to either overcome the surface tension or pull the entire droplet. The effect of droplet volume on SET operation was examined while keeping the SET diameter constant (FIG. 13B). At a given speed, larger droplets tended to facilitate particle extraction, and larger amount of MPs tended to promote droplet dispensing. This can be explained by the fact that, with all else constant, drag resistance increases with droplet volume, thus causing NP2 to yield first; Fₘ increases with MPs amount, thus causing NP1 to yield first. To visualize these results, we have constructed a 3D phase diagram by qualitatively summarizing our observations (FIG. 13C).

By controlling the MPs amount and droplet size, the same SETs can serve different functions. A good example is in the integrated genetic detection chip (FIG. 10). The MPs used for DNA isolation were relatively numerous. As they dragged the eluent droplet over the three small SETs, daughter droplets were formed (FIG. 10C), and subsequently PCR reaction buffer droplets were moved to mix with each of these eluent aliquots (FIG. 10D). Due to relatively few MPs used to actuate the reaction buffer droplet, the same SETs were able to immobilize the entire droplet and facilitate the MPs extraction (FIG. 10E). In this experiment, SETs size and MPs amount were selected to ensure efficient and reliable droplet operations. Because this often meant designing the system so that one necking point developed significantly faster than the other, only one neck point was observed in many cases.

Currently, a typical bench-top assay involving liquid handling includes removing, transferring, mixing, metering, and making aliquots, all in separate containers. However, SETs offers an alternative approach, because these procedures can be directly translated onto a SETs-enabled magnetic digital microfluidic platform. Liquids form droplets on the surface of the chip, which function as self-contained reaction chambers. Liquid transfer is realized by droplet transport, and liquid mixing is achieved by fusion. Waste liquid removal is accomplished by MPs extraction from the droplet using SETs.

More importantly, as we have demonstrated, SETs can be used to accurately meter and make liquid aliquots, which is not possible with traditional magnetic droplet platforms. As a result, SETs-enabled digital microfluidic platforms are able to translate all of the liquid operations required for assay preparation into the droplet format, thus allowing complex tasks with microliter volumes, and providing a versatile system for portable molecular sensing. With SETs, we have developed several droplet assay platforms with functions that may not be realized with traditional droplet systems. We advocate that SETs can significantly enhance the functionality, and broaden the applicability, of magnetic droplet platforms to numerous fields of life science.

### Materials and Methods

SU8 Shadow Masks Fabrication and SETs Patterning.

The SU8 shadow mask was lithographically defined, with a layer of TEFLON AF as the sacrificial layer using a lift-off process. A piece of glass slide of 3 mm thick was first cleaned with O₂ plasma, and then 1% w/w TEFLON AF (T_{g}=160° C.) dissolved fluorinated oil (Fluorinert FC-40) was spun on the substrate at 500 rpm, resulting in a layer of TEFLON AF approximately 120 nm thick. The thickness of the TEFLON AF layer was estimated by measuring the spectral reflectance using Filmetrics F20. The relationship between the thickness of the TEFLON AF nanofilm and spin speed was plotted. The TEFLON AF coated glass substrate was then treated with O₂ plasma for 5 s to improve the wettability for better photoresist adhesion (31). The SU8 3050 photoresist was spin-coated at 2000 rpm. The relationship between the thickness of SU8 3050 on the O₂ plasma-treated TEFLON AF nanofilm versus spin speed is shown in FIG. 3D. After UV exposure with a dosage of 200 nmJ/cm², the SU8 membrane, which served as shadow mask, was lifted off in the SU8 developer, cleaned with isopropanol alcohol (IPA), and dried with compressed air (FIG. 3B).

To pattern SETs on TEFLON nanofilm, 1% w/w TEFLON AF was coated on a piece of grade 1 glass coverslip at 2000 rpm. The SU8 shadow mask was then laminated on the coverslip and fixed with clamps. By subjecting the substrate to O₂ plasma at 325 mTorr and 50 mW for 40 s, the exposed TEFLON AF nanofilm was removed, and the area protected by the SU8 shadow mask remained intact. Because TEFLON AF nanofilm disintegrated when exposed to water right after O₂ plasma etching, we made a final step of baking the coverslip at 400° C. for 30 s in order to anneal the TEFLON AF nanofilm, and to promote its adhesion to the glass. The entire SETs patterning process was completed in 3 min when using the ready-made SU8 shadow mask.

Droplet Manipulation on SETs-Enabled Platform.

MPs (MagAttract Suspension G, Qiagen) were washed with water, dried, and added to droplets as the motion actuator. The density of the MPs was estimated by measuring the dried weight of 200 μL particle suspension. An N52 grade cylindrical neodymium permanent magnet (Diameter×Length=⅜″×¼″) was placed beneath the substrate for MPs actuation. The permanent magnet was either controlled manually or by a motorized translational stage.

In the experiment presented in FIGS. 8A and 8B, the SET had an outer diameter of 4 mm. A 0.12 mg aliquot of MPs was added to the orange droplet for actuation. In the experiments shown in FIG. 8C, the SETs were 2 mm in diameter and 0.36 mg of MPs was used to drive the droplets. 2 mm SETs were used in the experiment presented in FIG. 8D, and 4 mm SETs were used in experiment presented in FIG. 8E. The representative droplet array presented in FIG. 9B was generated on an array of SETs of 1.5 mm, 2 mm, and 2.5 mm in diameter. In all cases, no visible MPs were left in the SETs. To create the 3D phase diagram, we tested the SETs operations under the combined conditions of 9 different droplet volumes ranging from 5 μL to 100 μL, 12 different SETs sizes ranging from 0.25 mm to 5 mm in diameter and 12 different particle amounts ranging from 0.05 mg to 6 mg. The motion of the magnet was kept at a constant low speed of 0.5 mm/s using the translational stage.

Estimating Droplet Volume Dispensed by SETs.

Droplets containing 100 nM fluorescein were dispensed by SETs from the stock droplet. 10 μL of water was added to each dispensed daughter droplet. The volumes of the daughter droplets dispensed by SETs were calculated based on the relative change in fluorescent intensities according to Equation 1.

$${V}_{d} = \frac{{C}_{f}}{{C}_{i} - {C}_{f}} {V}_{{H}_{2} O} \qquad ( 1 )$$

where Cᵢ and C_{f} are the initial and final fluorescein concentrations respectively measured by fluorescent intensities, V_{d} is the volume of the daughter droplet and V_{H2O} is the volume of water added.

SETs-Enabled Integrated Sample Preparation and Genetic Detection.

All chemicals and reagents were acquired from Sigma-Aldrich unless otherwise stated. 10 μL of human whole blood was incubated with the lysis/binding buffer droplet containing 10 μL lysis/binding buffer (Buffer AL, Qiagen), 1.5 μL Qiagen protease (Qiagen), 10 μL IPA and 0.18 mg MPs (MagAttract Suspension G, Qiagen). One 35 μL droplet of washing buffer A (Buffer AW1, Qiagen) and two 20 μL droplets of washing buffer B (Buffer AW2, Qiagen) were dispensed onto the designated SETs. The DNA was eluted in a 5 μL elution buffer droplet containing 10 mM Tris at pH=8. The reaction buffer droplet contained 67 mM Tris, 16.6 mM ammonium sulfate, 6.7 mM MgCl₂, 10 nM 2-mercaptoethanol, 1 mM of each dNTP (GE healthcare), 800 dnM of each primer (Integrated DNA Technology), 0.1 U/μL Taq polymerase (Qiagen). The fluorescent signals were either generated from the Taqman probe (FIG. 10G) or 1×DNA intercalating dye EvaGreen (Biotium Inc.) (FIG. 10H). All droplets were submerged under mineral oil during the sample preparation process. After the reaction buffer droplets merged with the eluent droplets, the coverslip was carefully snapped along a scribed line on the backside. The part with reaction droplets were covered by mineral oil in a polydimethylsiloxane (PDMS) chamber with a glass bottom. The chamber was subjected to thermal cycling. The primer sequences and thermal cycling conditions are provided in Table 1 and Table 2.

TABLE 2
PCR conditions.

| Step | Temperature | Time | | Cycle number |
|---|---|---|---|---|
| Pre-denaturation | 95° C. | 5 | min | 1 |
| denaturation | 95° C. | 30 | s | 35 (for end point scan) |
| annealing | 58° C. | 30 | s | 50 (for real time) |
| elongation | 70° C. | 30 | s | |
| extended elongation | 70° C. | 5 | min | 1 |

FlipDrop Array.

0.36 mg of MPs was dried before being mixed with 10 μLdroplets. A magnet array consisted of N52 grade cylindrical neodymium permanent magnet (Diameter×Length=⅛″×½″) were fixed on a steel plate. All the stock droplets were driven at the same time using the magnet array over an array of SETs of 2 mm in diameter. Once the two droplet arrays were generated, a spacer made of PDMS was placed between the two arrays before flipping for droplet merging. The fluorescent images were scanned using Typhoon™ 9400 variable imager. To acquire QD signals, we used 488 nm laser, 610BP30 emission filter, 500PMT gain and 50 μM resolutions. To acquire Cy5 signals through FRET, we selected 488 nm laser, 670BP30 emission filter, 500PMT gain and 50 μM resolutions. The averaged intensities of each droplet from both scans were analyzed using ImageQuant™. The FRET factor was calculated based on Equation 2.

$$R = \frac{{I}_{a}}{{I}_{a} + {I}_{d}} \qquad ( 2 )$$

where R is the FRET factor, Iₐ and I_{d} are the average intensities of the FRET acceptor and donor respectively.

Dilution Series on SETs.

To create dilution series, SETs first metered a series of droplets of specific volumes from the stock solution droplet. The volume of the daughter droplets dispensed by SETs was calculated using Equation 3.

$${V}_{n} = \frac{m}{Dm + D - 1} {V}_{n - 1} \qquad ( 3 )$$

where Vₙ and Vₙ₋₁ are the volumes of the n^{th} and the (n−1)^{th} droplets respectively. The 1^{st} droplet is defined as the one with highest concentration in the dilution series. D is the dilution factor and m is the volume ratio of the dilution buffer droplet to the (n−1)^{th} droplet. The calculated droplet volume was subsequently mapped to the SETs size using the calibration curve (FIG. 10A). The twofold dilution series was made from 100 nM fluorescein. A 10 μL water droplet was used as dilution buffer. The initial and final fluorescent intensities were measured using NanoDrop 3300 fluorospectrometer.

Antibiotics Susceptibility Test.

A twofold dilution series was created from ampicillin stock solution on SETs. 10 μL of diluted Escherichia coli in LB broth was used as the dilution buffer. A control droplet containing no ampicillin was included. After 24 hr incubation in a humid chamber at 37° C., the optical density (OD) of the droplets was measured using NanoDrop 2000UV-Vis spectrometer. The measured ODs were normalized to the initial ODs measured before the incubation and the control droplet without ampicillin.

### REFERENCES

1. Daw, R. & Finkelstein, J. (2006) Nature 442, 367-367.
2. deMello, A. J. (2006) Nature 442, 394-402.
3. El-Ali, J., Sorger, P. K. & Jensen, K. F. (2006) Nature 442, 403-411.
4. Janasek, D., Franzke, J. & Manz, A. (2006) Nature 442, 374-380.
5. Yager, P., Edwards, T., Fu, E., Helton, K., Nelson, K., Tam, M. R. & Weigl, B. H. (2006) Nature 442, 412-418.
6. Abdelgawad, M. & Wheeler, A. R. (2009) Advanced Materials 21, 920-925.
7. Fair, R. B. (2007) Microfluidics and Nanofluidics 3, 245-281.
8. Pollack, M. G., Parnula, V. K., Srinivasan, V. & Eckhardt, A. E. (2011) Expert Review of Molecular Diagnostics 11, 393-407.
9. Cho, S. K. & Moon, H. (2008) Biochip Journal 2, 79-96.
10. Cho, S. K., Moon, H. J. & Kim, C. J. (2003) Journal of Microelectromechanical Systems 12, 70-80.
11. Lee, J., Moon, H., Fowler, J., Schoellhammer, T. & Kim, C. J. (2002) Sensors and Actuators a-Physical 95, 259-268.
12. Pollack, M. G., Fair, R. B. & Shenderov, A. D. (2000) Applied Physics Letters 77, 1725-1726.
13. Wheeler, A. R. (2008) Science 322, 539-540.
14. Pipper, J., Inoue, M., Ng, L. F. P., Neuzil, P., Zhang, Y. & Novak, L. (2007) Nature Medicine 13, 1259-1263.
15. Pipper, J., Zhang, Y., Neuzil, P. & Hsieh, T. M. (2008) Angewandte Chemie-International Edition 47, 3900-3904.
16. Zhang, Y., Park, S., Liu, K., Tsuan, J., Yang, S. & Wang, T.-H. (2011) Lab on a Chip 11, 398-406.
17. Rida, A., Fernandez, V. & Gijs, M. A. M. (2003) Applied Physics Letters 83, 2396-2398.
18. Guttenberg, Z., Muller, H., Habermuller, H., Geisbauer, A., Pipper, J., Felbel, J., Kielpinski, M., Scriba, J. & Wixforth, A. (2005) Lab on a Chip 5, 308-317.
19. Fan, S. K., Hsieh, T. H. & Lin, D. Y. (2009) Lab on a Chip 9, 1236-1242.
20. Sista, R., Hua, Z., Thwar, P., Sudarsan, A., Srinivasan, V., Eckhardt, A., Pollack, M. & Pamula, V. (2008) Lab on a Chip 8, 2091-2104.
21. Wang, Y., Zhao, Y. & Cho, S. K. (2007) Journal of Micromechanics and Microengineering 17, 2148-2156.
22. Zhao, Y., Yi, U.-C. & Cho, S. K. (2007) Journal of Microelectromechanical Systems 16, 1472-1481.
23. Ng, A. H. C., Choi, K., Luoma, R. P., Robinson, J. M. & Wheeler, A. R. (2012) Analytical Chemistry 84, 8805-8812.
24. Long, Z., Shetty, A. M., Solomon, M. J. & Larson, R. G. (2009) Lab on a Chip 9.
25. Shikida, M., Takayanagi, K., Inouchi, K., Honda, H. & Sato, K. (2006) Sensors and Actuators B: Chemical 113, 563-569.
26. Du, W., Li, L., Nichols, K. P. & Ismagilov, R. F. (2009) Lab on a Chip 9, 2286-2292.
27. Liu, J., Hansen, C. & Quake, S. R. (2003) Analytical Chemistry 75, 4718-4723,
28. Bailey, V. J., Easwaran, H., Zhang, Y., Griffiths, E., Belinsky, S. A., Herman, J. G., Baylin, S. B., Carraway, H. E. & Wang, T. H. (2009) Genome Research 19, 1455-1461.
29. Bailey, V. J., Keeley, B. P., Zhang, Y., Ho, Y. P., Easwaran, H., Brock, M. V., Pelosky, K. L., Carraway, H. E., Baylin, S. B., Herman, J. G. & Wang, T. H. (2010) Chembiochem 11, 71-4.
30. Zhang, C. Y., Yeh, H. C., Kuroki, M. T. & Wang, T. H. (2005) Nature Materials 4, 826-831.
31. Cho, C. C., Wallace, R. M. & Filessesler, L. A. (1994) Journal of Electronic Materials 23, 827-830.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A method of producing a fluidic chip, comprising:
providing a substrate having a first surface with a first free energy;
coating said first surface of said substrate with a layer of a second material having a surface with a second free energy;
arranging a shadow mask over said layer of said second material, said shadow mask defining a pattern of apertures; and
etching portions of said layer of said second material through said pattern of apertures to expose underlying portions of said first surface of said substrate such that said fluidic chip has a pattern of localized first free energy regions surrounded by said second material to provide surface energy traps for droplets of fluid,
wherein said first free energy differs in value from said second free energy.

2. A method of producing a fluidic chip according to claim 1, further comprising repeating said providing, coating, arranging and etching a plurality of times, sequentially, in that order, to produce a corresponding plurality of fluidic chips,
wherein said shadow mask used in said arranging is the same shadow mask reused for producing said plurality of fluidic chips.

3. A method of producing a fluidic chip according to claim 1, wherein said second free energy is greater than said first free energy.

4. A method of producing a fluidic chip according to claim 1, wherein said first surface of said substrate is a hydrophilic surface and said layer of second material is a hydrophobic surface.

5. A method of producing a fluidic chip according to claim 1, wherein said substrate is a glass substrate and said layer of second material is a layer of polytetrafluoroethylene.

6. A method of producing a fluidic chip according to claim 1, wherein said etching is an O₂ plasma etching.

7. A method of producing a fluidic chip according to claim 1, wherein said shadow mask comprises a photoresist material.

8. A method of producing a fluidic chip according to claim 7, further comprising producing said shadow mask by a photolithographic process prior to said arranging.

9. A method of producing a fluidic chip according to claim 1, wherein said shadow mask comprises a plurality of apertures patterns such that different aperture patterns can be selected during said arranging to provide selectable patterns of surface energy traps.

10. A method of using a fluidic device, comprising:
providing a fluidic chip having a plurality of surface energy traps;
depositing at least one fluid droplet on said fluidic chip, said fluid droplet comprising magnetic particles suspended therein; and
moving at least a portion of said at least one fluid droplet across a surface of said fluidic chip by altering a magnetic interaction applied to said magnetic particles such that said at least one fluid droplet interacts with at least one of said plurality of surface energy traps.

11. A method of using a fluidic device according to claim 10, wherein said moving at least a portion of said at least one fluid droplet across a surface of said fluidic chip comprises moving to a selected surface energy trap.

12. A method of using a fluidic device according to claim 11, wherein said surface energy trap has a second fluid droplet such that said moving causes said at least a portion of said at least one fluid droplet to merge with said second droplet.

13. A method of using a fluidic device according to claim 10, wherein said moving at least a portion of said at least one fluid droplet across a surface of said fluidic chip comprises moving a portion of said at least one fluid droplet out of a surface energy trap while leaving a portion of said at least one fluid droplet in said surface energy trap.

14. A method of using a fluidic device according to claim 10, wherein said moving at least a portion of said at least one fluid droplet across a surface of said fluidic chip comprises moving a portion of said at least one fluid droplet out of a surface energy trap while leaving a portion of said at least one fluid droplet in said surface energy trap to provide splitting of said at least one fluid droplet.

15. A method of using a fluidic device according to claim 10, wherein said moving at least a portion of said at least one fluid droplet across a surface of said fluidic chip comprises moving said at least one fluid droplet across a plurality of surface energy traps to split said at least one fluid droplet. a plurality of times leaving sub-droplets at each of said plurality of surface energy traps.

16. A method of using a fluidic device according to claim 10, further comprising:
providing a second fluidic chip having a second plurality of surface energy traps;
contacting a surface energy trap of said second fluidic chip with said at least one fluid droplet on the first-mentioned fluidic chip; and
removing said at least one fluid droplet from the first-mentioned fluidic chip by said second fluidic chip.

17. A method of using a fluidic device according to claim 10, further comprising:
providing a second fluidic chip having a second plurality of surface energy traps and at least one second fluid droplet held by one of said second plurality of surface energy traps;
contacting said at least one second fluid droplet with said at least one fluid droplet on the first-mentioned fluidic chip to cause said droplets to form a merged droplet; and
removing said second fluidic chip leaving said merged droplet attached to one of the first-mentioned or the second fluidic chip.

18. A fluidic chip, comprising:
a glass substrate; and
a layer of polytetrafluoroethylene on a surface of said glass substrate,
wherein said layer of polytetrafluoroethylene defines a pattern of apertures therethrough to expose surface portions of said glass substrate so as to provide a pattern of surface energy traps.
